(19) United States
(12) Patent Application Publication
(10) Pub. No.: US 2004/0220667 A1
(43) Pub. Date: Nov. 4, 2004

(54) Implantable device using diamond-like carbon coating

(76) Inventors: Vladimir Gelfandbein, Pleasanton, CA (US); George Y. McLean, Palo Alto, CA (US)

Correspondence Address: BRINKS HOFER GILSON & LIONE, P.O. BOX 10395, CHICAGO, IL, 60610, US

(21) Appl. No.: 10/774,333

(22) Filed: Feb. 6, 2004

Related U.S. Application Data
(60) Provisional application No. 60/445,637, filed on Feb. 7, 2003.

(51) Int. Cl. A61L 2/00; B05D 3/00; A61F 2/16

(52) U.S. Cl. 623/6.56; 427/2.24; 623/901

(57) ABSTRACT

Diamond-like carbon films are deposited on devices for implantation in tissues of a living body, preferably, retinal implants. Openings may be formed in the diamond-like carbon film, preferably in alignment with portions of the device intended for electrical contact with surrounding tissues, i.e., electrodes. Alternatively, the diamond-like carbon film may be rendered electrically conductive. Furthermore, the diamond-like carbon films may be created in such a manner that they are substantially transparent to various wavelengths of electromagnetic radiation, including visible and/or infrared light. In a presently preferred embodiment, the diamond-like carbon film is deposited using a magnetically-filtered, cathodic arc physical vapor deposition process. Implantable devices, particularly retinal implants, comprising a diamond-like carbon film may exhibit excellent biocompatibility and biodurability properties in comparison with prior art devices and passivation coatings.

### CROSS-REFERENCE TO RELATED APPLICATIONS

The instant application claims the benefit of Provisional U.S. Patent Application Serial No. 60/445,637, entitled “Subretinal Implant Using Diamond-Like Films” and filed Feb. 7, 2003, the entirety of which is incorporated by reference herein.

### FIELD OF THE INVENTION

The present invention generally relates to devices that may be implanted in living tissue and, in particular, to implantable devices, including retinal implants, coated with diamond-like carbon.

### BACKGROUND

Modern medical technology has led to the creation of devices capable of implantation in tissues of a living body, e.g., pacemakers, bone implants, stents, etc. A challenge typically surrounding such devices is the need to render them biocompatible such that tissues surrounding these devices are not adversely affected. The nature of the material used to build these devices often leads to the requirement that the devices be hermetically sealed save for a selected portion of the device intended for direct communication with the surrounding tissues, e.g., stimulating electrodes. On the other hand, such devices must exhibit good biodurability; that is, the ability to stand up to the often chemically-aggressive environment exhibited by the tissues of the body.

To this end, there are numerous materials (sometimes referred to as passivation materials) known in the art that are used to substantially encase implantable devices to render them substantially biocompatible and biodurable, including various metals, alloys, plastics, ceramics, etc. A particular challenge in selecting a suitable passivation material arises in the case of very small, electrically active devices such as retinal implants. Examples of such devices include sub-retinal implants of the type being developed by Optobionics Corporation or epi-retinal implants of the type being developed by Second Sight LLP. Both sub-retinal and epi-retinal implants come into contact with the exceedingly delicate and sensitive tissues of the retina, as well as the chemically harsh saline environment of the eye. In these types of implants, it is known to use silicon dioxide or flexible polymers as passivation materials. The use of thin films of ultra-nanocrystalline diamond (UNCD) as a passivation material for implantable devices, including retinal implants, is described in U.S. Patent Application Publication No. 2002/0120296. UNCD is one example of a carbon-based, polycrystalline material that exhibits durability and chemical inertness characteristics similar to natural diamond. However, other carbonaceous materials, such as substantially amorphous diamond-like carbon films, likewise exhibiting diamond-like properties, have been recently developed and offer promise as passivation materials.

### BRIEF SUMMARY

The present invention describes the use of diamond-like carbon films deposited on devices for implantation in tissues of a living body. In a presently preferred embodiment, retinal implants are provided with a film of diamond-like carbon deposited on at least a portion thereof. Openings may be formed in the diamond-like carbon film. Where the implantable device is electrically active, such openings are preferably aligned with portions of the device intended for electrical contact with surrounding tissues, i.e., electrodes. Alternatively, the diamond-like carbon film may be rendered electrically conductive thereby obviating the need to create openings in the film. Furthermore, the diamond-like carbon films may be created in such a manner that they are substantially transparent to various wavelengths of electromagnetic radiation, including visible and/or infrared light. In a presently preferred embodiment, the diamond-like carbon film is deposited using a magnetically-filtered, cathodic arc physical vapor deposition process. Implantable devices, particularly retinal implants, comprising a diamond-like carbon film may exhibit excellent biocompatibility and biodurability properties in comparison with prior art devices and passivation coatings.

### BRIEF DESCRIPTION OF THE DRAWINGS

FIG. 1 is a schematic, cross-sectional view of an exemplary implant in accordance with the present invention.

FIG. 2 is a schematic, cross-sectional view of an exemplary implant in accordance with an alternate embodiment of the present invention.

FIG. 3 is a schematic, cross-sectional detail view of an alternative electrode structure in accordance with the present invention.

FIG. 4 is a schematic illustration of a preferred system for depositing diamond-like carbon films in accordance with the present invention.

### DETAILED DESCRIPTION OF THE PRESENTLY PREFERRED EMBODIMENTS

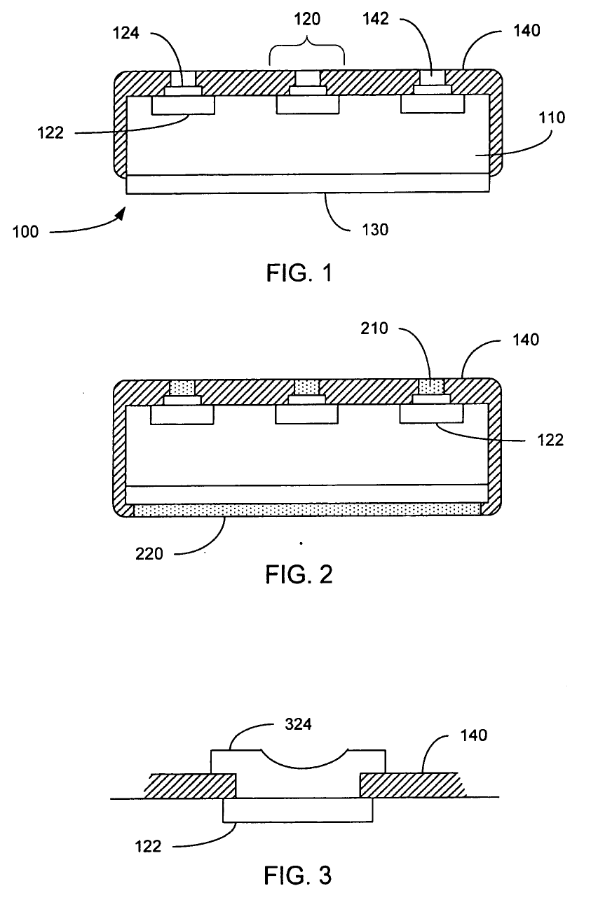

Referring now to FIG. 1, a device 100 having a diamond-like carbon (DLC) film 140 deposited on at least a portion of the device is illustrated. In the particular embodiment shown, the device 100 is a sub-retinal type device such as those manufactured by Optobionics Corporation for the treatment of degenerative retinal diseases, an example of which is further described in U.S. Pat. No. 5,016,633, assigned to Optobionics Corp., and incorporated herein by this reference. However, it is understood that the present invention may be applied to a wide variety of retinal implant devices (including epi-retinal devices), specifically any devices designed to stimulate a retina of an eye (though not necessarily through direct contact with the retina) and that may benefit from the application of a passivation layer. Additionally, the devices to be coated with the DLC film are preferably electrically active devices capable, once implanted, of applying electrical stimulation to, or sensing electrical activity within, surrounding tissues. When intended for operation on a very small scale, as in the case of retinal implants, such devices may be fabricated using well-known semiconductor processing technology.

The device 100 comprises a substrate 110, which may be fabricated from silicon or a compound semiconductor material, in which one or more photovoltaic devices 120 are formed. Where semiconductor materials are used, the substrate may be positively or negatively doped. Of course, it is understood that material systems other than semiconductor material systems may be equally employed to implement suitable photovoltaic devices. Note that, for ease of illustration, only a small number of photovoltaic devices 120 are shown; in practice, a greater or lesser number of photovoltaic devices could be employed. Additionally, the dimensions shown in FIG. 1 are not to scale. Generally, each photovoltaic device 120 comprises an electrically active region 122, typically formed from positively and/or negatively doped regions, deposited in a front or top surface of the substrate 110. An electrode structure 124 is formed in electrical communication with the electrically active region 122 to facilitate transfer of electrical charge to tissue surrounding the device when implanted. As known in the art, each electrode structure 124 may be fabricated from various materials exhibiting good biocompatibility, biodurability and charge transfer characteristics, such as platinum, titanium, iridium or alloys or compounds thereof. In a presently preferred embodiment, each electrode structure 124 comprises a layer of iridium oxide overlying a bonding layer of titanium. Additionally, a common or ground electrode 130, also preferably fabricated from iridium oxide overlying a titanium bonding layer, is formed on a back or bottom surface of the substrate 110.

As shown, a DLC film 140 is deposited on at least a portion of the underlying device 100. As known in the art, DLC is ideally one form of carbon (or, sometimes in practice, hydrogenated carbon) that may be generally distinguished from other carbonaceous compounds based on the atomic bonding structure and crystalline nature of each material. For example, natural diamonds are typically formed as a single-orientation crystal or polycrystalline structure comprised almost exclusively of sp³ carbon bonds. In contrast, amorphous materials such as DLC are non-crystalline, having virtually no long-range order or well-defined periodic structure, and are formed from a mixture of carbon atoms (and possibly hydrogen atoms or other dopants) exhibiting not only sp³ bonds, but also a significant proportion of sp² bonds. Specific properties of such amorphous materials may be controlled by altering the sp³:sp² bond ratio. Further still, other materials, such as the UNCD described in U.S. patent application Publication No. 2002/0120296, are polycrystalline in nature exhibiting relatively little sp² bond content. As the name would imply, polycrystalline materials are formed of numerous small regions (grains) of periodic, variously-oriented crystalline structures separated by grain boundaries. Depending on the process used to form a polycrystalline material, individual grain sizes may vary from relatively large (1-10 micrometer diameters) to very small (2-5 nanometer diameters). Simply put, polycrystalline films are comprised of many variously-oriented diamond crystals, whereas DLC films demonstrate substantially no crystallinity while still exhibiting numerous diamond-like properties. While both amorphous and polycrystalline carbonaceous materials can exhibit properties similar to natural diamond, a significant difference between the two types of materials is the cost and complexity of production, with amorphous materials being simpler and less expensive. For example, the UNCD described in U.S. patent application Publication No. 2002/0120296 is deposited using a microwave plasma chemical vapor deposition formed in an atmosphere of argon and carbon or hydrocarbon constituents. In contrast, as described in greater detail below, a preferred technique for depositing DLC films in accordance with the present invention obviates the need for carefully controlled atmospheres through the use of a comparatively simple vacuum cathodic arc plasma physical vapor deposition technique.

Generally, the DLC film 140 may be from about 5 to 150 nanometers thick, with a preferred thickness in the range of about 75-100 nanometers thick. Note that uniform thickness of the DLC film is preferred, but not a requirement. In general, the upper limits of these ranges are governed by several factors. For example, thicker DLC films typically give rise to greater internal stresses which can lead to deformation of the device or delamination of the DLC film. Delamination is further controlled by the relative adherence of the DLC film to the underlying substrate. In accordance with the present invention, the DLC film preferably adheres well with either a silicon or silicon oxide surface.

Optical transparency, another factor regarding thickness, may be adversely affected by thicker films. Further still, greater deposition times are required for thicker films. Thus, the upper bound of DLC film thickness is a matter of design choice dependent upon the above-mentioned performance and manufacturing parameters.

Stepped structures, such as alternating layers of structurally different DLC films, may be used to control stresses that would otherwise arise in continuous DLC films of equivalent overall thickness. For example, one or more layers of DLC having relatively high sp³:sp² bond ratios may be interleaved with one or more layers of DLC having relatively low sp³:sp² bond ratios. In a similar vein, stresses may also be controlled through a graded, as opposed to discretely layered, film in which the structural characteristics of the film are varied as the thickness of the film increases. For example, as the thickness of the film increases, the sp³:sp² bond ratio may likewise be increased in a continuous fashion such that the resulting film is more graphitic in nature at its base, smoothly transitioning to more diamond-like at its outer surface. Whereas DLC films deposited using other techniques normally have high intrinsic stress (typically in the range of 1.0-1.5 GPa), DLC films formed in accordance with the presently preferred cathodic arc plasma PVD technique may be designed to have internal stresses reduced to the point of being difficult to measure.

As illustrated in FIG. 1, openings 142 may be formed in the DLC film 140 using known etching techniques. Additionally—unlike most CVD or PECVD techniques that are performed at higher temperatures—well-known temperature-sensitive lift-off techniques may be employed when forming such openings in the DLC film. Preferably, where the underlying device is electrically active, such openings 142 are formed in those regions of the DLC film 140 overlying the electrode structures 124 such that the electrode structures 124 are exposed to the surrounding environment.

In FIG. 1, the DLC film 140 is not deposited over the common electrode 130, i.e., only a portion of the device is covered. However, the present invention is not so limited and may include devices that are substantially totally encased in a DLC film. Such a total encasement approach may offer better hermetic properties in some applications. This is illustrated in FIG. 2 where the DLC layer 140 entirely envelopes the device. In these configurations, it may be advantageous to further render portions of the DLC film 140 electrically conductive. Such modifications can be made to the DLC film 140 through selective doping, as known in the art. This is illustrated in FIG. 2 by electrically conductive portions 210, 220 formed in the DLC film 140 overlying the electrode structures and/or common electrode.

In yet another embodiment, illustrated in FIG. 3, an alternate electrode structure 324 may be fabricated. In this embodiment, the DLC film 140 can be deposited directly on the electrically active region 122. Thereafter, an opening is formed in the DLC film 140 above the electrically active region 122 (using, for example, a suitable etching or lift-off technique) and the electrode structure 324 is formed in the opening using known techniques. As in the previous embodiment, the electrode structure 324 illustrated in FIG. 3 may comprise a suitable metallic bonding layer.

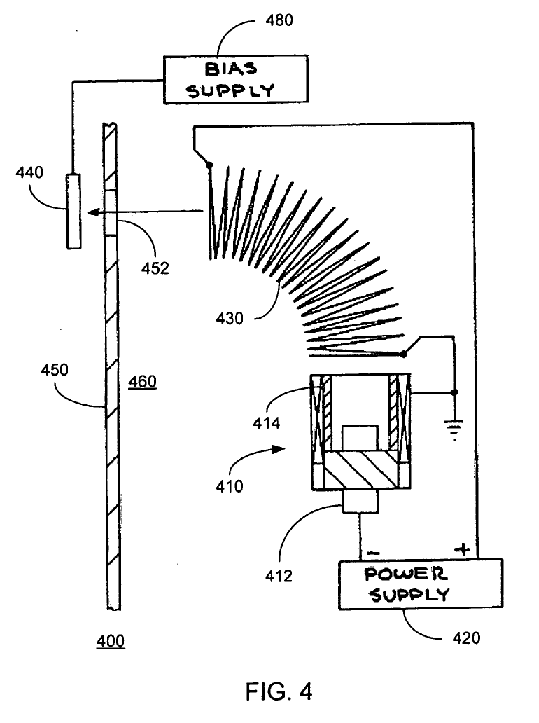

DLC films of the type used in the present invention may be fabricated using a variety of known techniques. For example, chemical vapor deposition (CVD), plasma enhanced chemical vapor deposition (PECVD) and physical vapor deposition (PVD, including ion-beam deposition or sputtering) are all well-known techniques used to create DLC films. Additionally, a magnetically filtered, vacuum (cathodic) arc plasma PVD technique has been developed. Such a technique is further described in U.S. Pat. No. 6,465,780 issued to Anders et al. (“the '780 patent”), the teachings of which are incorporated herein by reference. Generally, the '780 patent teaches a vacuum or cathodic arc plasma deposition system 400 (FIG. 4) including a plasma source 410 which itself comprises a cathode 412 and an anode 414. Operating within a vacuum and a filter chamber 460, the plasma source 410 generates plasma under the control of an arc current power supply 420. In a presently preferred embodiment, the cathode 412 comprises a movable rod of graphite that is consumed during the plasma generation process. As known in the art, such a cathodic arc-generated plasma will typically contain so-called macroparticles that, if deposited on a substrate 440, will result in a degraded film.

To combat this problem, prior to being directed to the substrate 440, the plasma enters a magnetic filter 430 that is charged to an opposite polarity (relative to the plasma source 410) by the power supply 420. The magnetic filter 430 preferably comprises an open-architecture, three-dimensional double-bent solenoid or twist filter that substantially filters out macroparticles or neutrally charged particles, resulting in high quality, particle-free plasma and, consequently, particle-free DLC films. As the plasma exits the filter 430, it is directed toward a substrate or target 440 that, in the context of the present invention, would comprise the implantable device. Further macroparticle filtering may be achieved by directing the plasma through an opening 452 in a macroparticle firewall 450 separating the target 440 from the filter chamber 460. As the plasma impacts the target 440, a DLC film is deposited. To further control the energy at which the plasma is directed at the target 44, and thereby further control the properties of the deposited DLC film, a bias power supply 480 may be coupled to the target. Further still, both the bias supply 480 and the arc current power supply 420 may operate in pulsed manners, as known in the art, to further refine the deposition of the DLC film.

Using the above-described cathodic arc plasma PVD technique, DLC films exhibiting substantial diamond-like properties, particularly with respect to biodurability and biocompatibility, may be fabricated. In part, this is a result of the comparatively high ratio of sp³ bonds that may be designed into such films. For example, using other CVD/PECVD methods, a sp³:sp² ratio of 30-50% is typically achieved. In contrast, DLC with sp³:sp² ratios of up to 85% are readily achievable using the presently preferred cathodic arc plasma PVD technique. Additionally, the significant corrosion resistance exhibited by the preferred DLC films results from the exceptionally low occurrence of so-called “pinholes” relative to DLC films of comparable thicknesses produced using other CVD/PECVD methods.

In one embodiment of the present invention, the DLC film can be fabricated in such a manner that the film exhibits varying degrees of transmittance to different wavelengths of electromagnetic radiation. For example, where the device to be coated comprises one or more photovoltaic devices (see FIGS. 1-3), the deposited DLC film is preferably fabricated to be substantially transparent to visible and/or infrared light wavelengths. Generally speaking, opacity of a DLC film increases in proportion to the thickness of the DLC film. Furthermore, opacity increases as DLC films become more graphitic.

The present invention describes the use of diamond-like carbon films on devices intended for implantation in living tissues. The use of DLC films increases the biocompatibility and biodurability of such devices, which devices may nevertheless remain in communication with their surrounding environments.

It is intended that foregoing detailed description should be regarded as illustrative rather than limiting, and that it be understood that the following claims, including all equivalents are intended to define the scope of this invention.

We claim:

1. A retinal implant comprising:
a device for implantation in an eye for stimulation of a retina of the eye; and
a diamond-like carbon film deposited on at least a portion of the device.

2. The retinal implant of claim 1, wherein the device is suitable for epi-retinal implantation.

3. The retinal implant of claim 1, wherein the device is suitable for sub-retinal implantation.

4. The retinal implant of claim 3, wherein the sub-retinal implant comprises at least one photovoltaic device.

5. The retinal implant of claim 1, wherein the diamond-like carbon film comprises at least one opening therein.

6. The retinal implant of claim 5, wherein the device comprises at least one electrode and the at least one opening in the diamond-like carbon film is aligned with the at least one electrode.

7. The retinal implant of claim 5, wherein at least one electrode is formed within the at least one opening in the diamond-like carbon film.

8. The retinal implant of claim 1, wherein at least a portion of diamond-like carbon film is electrically conductive.

9. The retinal implant of claim 1, wherein the diamond-like carbon film is substantially transparent to wavelengths of visible light.

10. The retinal implant of claim 1, wherein the diamond-like carbon film is substantially transparent to wavelengths of infrared light.

11. The retinal implant of claim 1, wherein the diamond-like carbon film comprises a plurality of structurally different diamond-like carbon films.

12. The retinal implant of claim 1, wherein the diamond-like carbon film comprises a structurally graded diamond-like carbon film.

13. A retinal implant provided by the process of:
providing a device for implantation in an eye for stimulation of a retina of the eye;
forming a carbonaceous cathodic arc plasma; and
directing the plasma to the device to deposit a diamond-like carbon film on at least a portion of the device.

14. The retinal implant of claim 13, wherein the process further comprises:
magnetically filtering the plasma prior to deposition of the diamond-like carbon film on the device.

15. The retinal implant of claim 13, wherein the process further comprises:
electrically biasing the device during deposition of the diamond-like carbon film on the device.

16. The retinal implant of claim 15, further comprising electrically biasing the device in a pulsed fashion.

17. The retinal implant of claim 13, wherein the process further comprises:
removing at least a portion of the diamond-like carbon film to create at least one opening therein.

18. The retinal implant of claim 13, wherein the process further comprises:
rendering at least a portion of the diamond-like carbon film electrically conductive.

19. A method for providing a retinal implant, the method comprising:
providing a device for implantation in an eye for stimulation of a retina of the eye; and
depositing a diamond-like carbon film on at least a portion of the device.

20. The method of claim 19, wherein depositing the diamond-like carbon film further comprises:
forming a carbonaceous cathodic arc plasma; and
directing the plasma to the device to deposit the diamond-like carbon film.

21. The method of claim 20, wherein depositing the diamond-like carbon film further comprises:
magnetically filtering the plasma prior to deposition of the diamond-like carbon film on the device.

22. The method of claim 19, wherein depositing the diamond-like carbon film further comprises:
electrically biasing the device during deposition of the diamond-like carbon film on the device.

23. The method of claim 22, further comprising electrically biasing the device in a pulsed fashion.

24. The method of claim 19, further comprising:
removing at least a portion of the diamond-like carbon film to form an opening therein.

25. The method of claim 19, further comprising:
rendering at least a portion of diamond-like carbon film electrically conductive.

26. The method of claim 19, wherein depositing the diamond-like carbon film further comprises depositing a diamond-like carbon film that is substantially transparent to wavelengths of visible light.

27. The method of claim 19, wherein depositing the diamond-like carbon film further comprises depositing a diamond-like carbon film that is substantially transparent to wavelengths of infrared light.
